(19) United States
(12) Patent Application Publication
(10) Pub. No.: US 2026/0058008 A1
(43) Pub. Date: Feb. 26, 2026

(54) CREW RESOURCE MANAGEMENT FOR CLINICAL GUIDANCE

(71) Applicant: CareFusion 303, Inc., San Diego, CA (US)

(72) Inventors: Daniel M. ABAL, San Diego, CA (US); Brendan John Burgess, Poway, CA (US)

(21) Appl. No.: 19/100,001

(22) PCT Filed: Aug. 3, 2023
(86) PCT No.: PCT/US2023/029366

§ 371 Date: Jan. 30, 2025

Related U.S. Application Data
(60) Provisional application No. 63/395,294, filed on Aug. 4, 2022.

(51) Int. Cl. G16H 40/63; G16H 20/00; G16H 40/20

(52) U.S. Cl. CPC G16H 40/63; G16H 40/20; G16H 20/00

(57) ABSTRACT

A method for medical device resource management includes monitoring a plurality of medical devices for adjustments to the medical devices during a period of time; identifying, based on the monitoring, the adjustments to the plurality of medical devices during the period of time; building a therapy profile associated with a patient and the medical devices based on the identified adjustments; identifying an adverse event pertaining the patient or a respective medical device of the plurality of medical devices; and after identifying the adverse event: generating, based on the therapy profile, a plurality of consecutive control signals to correct the adverse event, each being associated with a different one of the medical devices or a different user.

### REFERENCE TO RELATED APPLICATIONS

This application claims priority to U.S. Provisional Patent Application 63/395,294, filed Aug. 4, 2022, the entire disclosure of which is incorporated herein by this reference.

### FIELD OF THE INVENTION

The present disclosure is generally related to a control device configured facilitate crew resource management.

### BACKGROUND

Crew resource management (CRM) is a set of procedures for use in environments where human error can have devastating effects. CRM is primarily used for improving aviation safety and focuses on interpersonal communication, leadership, and decision making in aircraft cockpits. CRM has been shown to be very effective in managing flight emergencies and preventing disasters and loss of life.

### SUMMARY

CRM can be applied to the healthcare industry to address emergency issues. The use of checklists may provide a measure of error proofing during a medical emergency or other high-risk situations where knowing what steps to perform during a stressful time-sensitive situation may be very crucial, and relying on a clinician's memory may not be optimal.

Instead of using physical checklists (e.g., flip charts), which can be lost, or not easily available when needed, the subject technology described herein provides a crew resource management unit integrated with an intelligent control module (ICM). Unlike a checklist which cannot be readily updated when changes or new procedures are to be added, the CRM unit can receive timely and efficient updated procedural/protocol information via software updates performed over the institution's network, for example, without active user input, and actively make real time therapy decisions and/or adjustments based on that information. Traveling clinicians may not be familiar with emergency practices of a particular hospital, and thus are prone to making mistakes and/or being subject to unnecessary delays in clinical settings. Thus, the disclosed centrally managed CRM unit can synchronize the actions to be undertaken by different clinicians in an emergency situation by expressly providing protocol information to respective clinicians, effecting therapy changes, and reducing the amount of uncertainty and improving coordination.

By dynamically presenting relevant protocol information on a display of the ICM, or on a display of a user device of a respective clinician, a user interface can be presented to a clinician, or to a group of clinicians, with important information emphasized or highlighted. The interface and/or algorithms behind the interface may include and/or integrate calculators for determining a weight-based dosage of medication for a patient. In addition, information about medications (e.g., mixing ratios of different medications, compatibility of medications) and adverse effects can also be presented to a clinician, without the clinician having to locate and read from the instructions on the packaging of the medication. In addition, the subject technology enables a clinician to navigate to pertinent information more quickly by guiding the clinician(s) to the order of steps and information needed at the appropriate time. Additional resources (e.g., submitting pharmacy requests, summoning specialists for consult, requesting code alerts, requests for equipment, access to patient's electronic health records for critical health and allergy information) can be accessed by the communication capabilities of the ICM using the CRM program. The CRM can keep a record of the steps performed by different clinicians for later analysis.

According to various aspects, the subject technology provides a system and method for intelligent medical device resource management. In this regard, a medical device resource management system is disclosed for use with infusion pumps and related devices.

According to various aspects, a disclosed medical device resource management system includes a communication interface configured to exchange information with a plurality of medical devices, and to exchange information with at least one of a plurality of user devices; a processor; and a non-transitory computer readable medium including instructions that, when executed by the processor, cause the medical device resource management system to: monitor the plurality of medical devices for adjustments to the plurality of medical devices during a period of time; identify, based on the monitoring, the adjustments to the plurality of medical devices during the period of time; build a therapy profile associated with a patient and the plurality of medical devices based on the identified plurality of adjustments; identify an adverse event pertaining the patient or a respective medical device of the plurality of medical devices; responsive to identifying the adverse event: generate, based on the therapy profile, a plurality of consecutive control signals to correct the adverse event, each being associated with a different one of the medical devices or a different user.

According to various aspects, a disclosed method includes monitoring a plurality of medical devices for adjustments to the medical devices during a period of time; identifying, based on the monitoring, a plurality of the adjustments to the medical devices over the course of the period of time; building a therapy profile associated with a patient and the medical devices based on the identified plurality of adjustments; identifying an adverse event pertaining the patient or a respective medical device of the plurality of medical devices; responsive to identifying the adverse event: generating, based on the therapy profile, a plurality of consecutive control signals to correct the adverse event, each being associated with a different one of the medical devices or a different user. Other aspects include corresponding systems, apparatus (e.g., an infusion device), and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2 is a conceptual diagram illustrating an example system architecture, including an example crew resource management unit, according to aspects of the subject technology.

FIG. 3 is a conceptual diagram illustrating an example user interface for placing a code call, according to aspects of the subject technology.

FIG. 4 depicts an example process executed prior to beginning closed loop control, according to aspects of the subject technology.

FIG. 5 depicts an example selection panel, according to aspects of the subject technology.

FIG. 6 shows a selection catalog for other emergency conditions, according to aspects of the subject technology.

FIG. 7A depicts an example process for intelligently responding to an emergency clinical situation, according to aspects of the subject technology.

FIG. 7B depicts a subsequent portion of the example process shown in FIG. 7A, according to aspects of the subject technology.

FIG. 8A depicts an example process for intelligently responding to an emergency clinical situation, according to aspects of the subject technology.

FIG. 8B depicts a subsequent portion of the example process shown in FIG. 8A, according to aspects of the subject technology.

FIG. 9 depicts an example process medical device resource management, according to aspects of the subject technology

FIG. 10 is a conceptual diagram illustrating an example electronic system 1000 for crew resource management, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

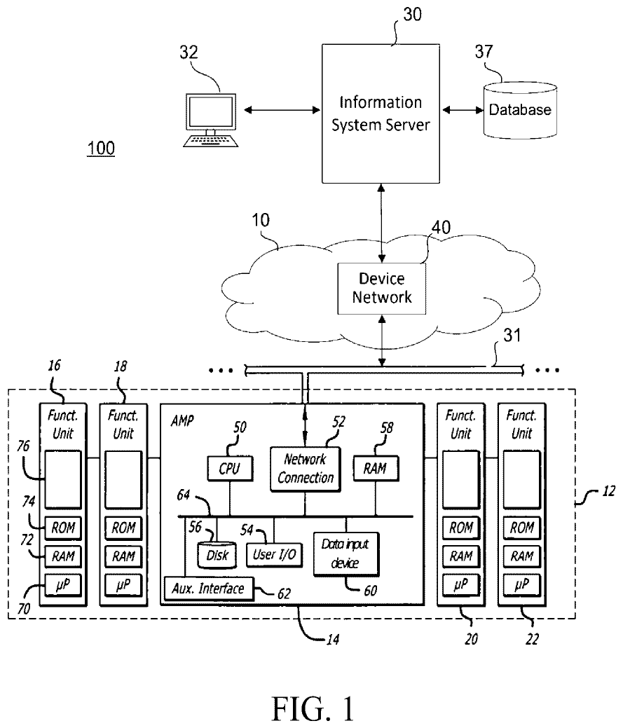

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 may be a wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistants, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a interface device 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include means (specifically configured with one or more features described) for displaying and inputting information, such as a monitor, a printer, a keyboard, softkeys, a mouse, a track ball and/or a light pen. Data input device 60 may be a bar code reader capable of scanning and interpreting data printed in bar coded format. Additionally, or in the alternative, data input device 60 can be a specifically configured device for entering coded data into a computer, such as a device(s) for reading a magnetic strip, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or other appropriate communication means. Auxiliary interface 62 may be an RS-232 communications interface, however other means for communicating with a peripheral device in the described environments such as a printer, patient monitor, infusion pump or other medical device may be used without departing from the subject technology. Additionally, data input device 60 may be a separate functional module, such as modules 16, 18, 20 and 22, and configured to communicate with controller 14, or other systems on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem, or a cable modem. Direct or indirect network connection may be used, including, but not limited to a telephone modem, an MIB system, an RS232 interface, an auxiliary interface, an optical link, an infrared link, a radio frequency link, a microwave link, a personal area network connection, a local area network connection, a cellular link, or a WLANS connection or other wireless connection.

Functional modules 16, 18, 20, 22 are specially configured devices for providing care to a patient or for monitoring patient condition. As shown in FIG. 1, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be a patient treatment or monitoring device including, but not limited to, an infusion pump, a syringe pump, a patient-controlled analgesia (PCA) pump, an epidural pump, an enteral pump, a blood pressure monitor, a pulse oximeter, an EKG monitor, an EEG monitor, a heart rate monitor, or an intracranial pressure monitor or the like. Functional module 18, 20 and/or 22 may be a printer, scanner, bar code reader or other peripheral input, output, or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1, any number of devices may be connected directly or indirectly to central controller 14. The number and type of functional modules described herein are intended to be illustrative, and in no way limit the scope of the subject technology. Module-specific components 76 include specifically configured components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics, or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction, or a combination of both automated and manual interaction. Automated interaction may be continuous or intermittent and may occur through direct network connection 54 (as shown in FIG. 1), or through RS232 links, MIB systems, RF links such as BLUETOOTH, IR links, PANS, LANS, WLANS, digital cable systems, telephone modems or other wired or wireless communication means. Manual between patient care device 12 and network 10 involves physically transferring, intermittently or periodically, data between systems using, for example, user interface device 54, coded data input device 60, bar codes, computer disks, portable data assistants, memory cards, or other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication

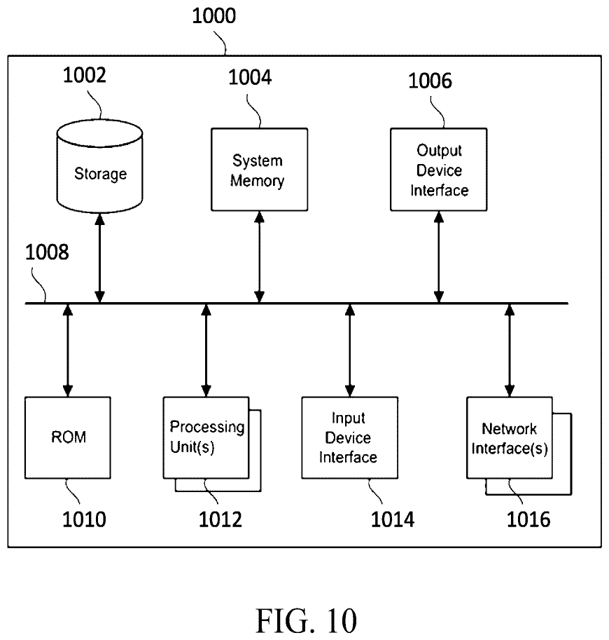

In some implementations, medication delivery modules 16, 18, 20, 22 include plug-in ports for expansion. Accordingly, a new medication delivery module may be attached to PCU 12 by coupling a connector through the plug-in ports, which may include electrical terminals so that the added medication delivery module 16, 18, 20, 22 may transmit and receive information to and from a control module 14. In some implementations, the added medication delivery module 16, 18, 20, 22 may also receive power from control module 14 through a plug-in port. Control module 14 may include a main display, a memory and a processor (see FIG. 10), and may be configured to display operational parameters and medication delivery status, and further information associated with each of medication delivery modules 16, 18, 20, 22. According to various implementations, module displays may also display physiological data (e.g., vital signs) associated with a patient.

A main display (e.g., I/O 54) may be configured to display one or more user interfaces for the display of operational parameters or other data associated with a module 16, 18, 20, 22, and/or physiological parameters associated with the patient. The main display may include multiple user interfaces, with each individual user interface graphically displaying information for a respective one of medication modules, including information also displayed on a corresponding module displays. In some implementations, control module 14 includes a communications module (including, e.g., an antenna), configured to communicate wirelessly with a controller, or with a network.

With reference to FIG. 1, when a medication delivery module 16, 18, 20, 22 initiates an infusion of a medication to the patient, the control module 14 is configured to create and manage an infusion session within a memory of the control module (or related module). For the purpose of this disclosure, the infusion session includes state information of the PCU 12, its control module 14, and/or its associated modules, which is recorded and saved to memory during a particular period of time. The state information includes, but is not limited to, records of parameter values utilized by the PCU, its control module, and/or its associated modules during the period of time, and/or records physiological data collected during the period of time. During the infusion, physiological data associated with the patient is recorded within the session, operating parameter values, and modifications to the operating parameters of the PCU, its control module, and/or modules are also recorded in the session.

If not already logged into the PCU 12, the clinician may scan his or her badge proximate to a sensor (e.g., 54, 60) on the PCU 12, and the PCU may attempt to authenticate the clinician by sending the clinician's scanned identification to server 30. The clinician's badge may incorporate a radio frequency identification device (RFID), which is read by a scanner integrated with the PCU, or a portable scanner associated with the PCU. The clinician may scan his or her badge at the control module 14 to identify and authorize the clinician to initiate the administration of a medication. Once the clinician is associated with the PCU and/or module(s), the clinician's identification is associated with the session. The same is applicable with a patient. The clinician may scan the patient's wristband with a portable scanner, or using the sensor on the PCU 14 (or its control module) to associate the patient with the PCU and/or module(s) (and a session).

The control unit 14 of PCU 12 is configured to generate a graphical representation of the infusion session, and display (e.g., in a display) the graphical representation, including a graphical visualization of all parameters of the infusion during the session and modifications to the parameters, together with physiological data obtained during the session. The graphical representation may include pseudo identifiers for unknown data until such data is substituted with known identifiers. At that time, the graphical representation is displayed with the known patient identifiers

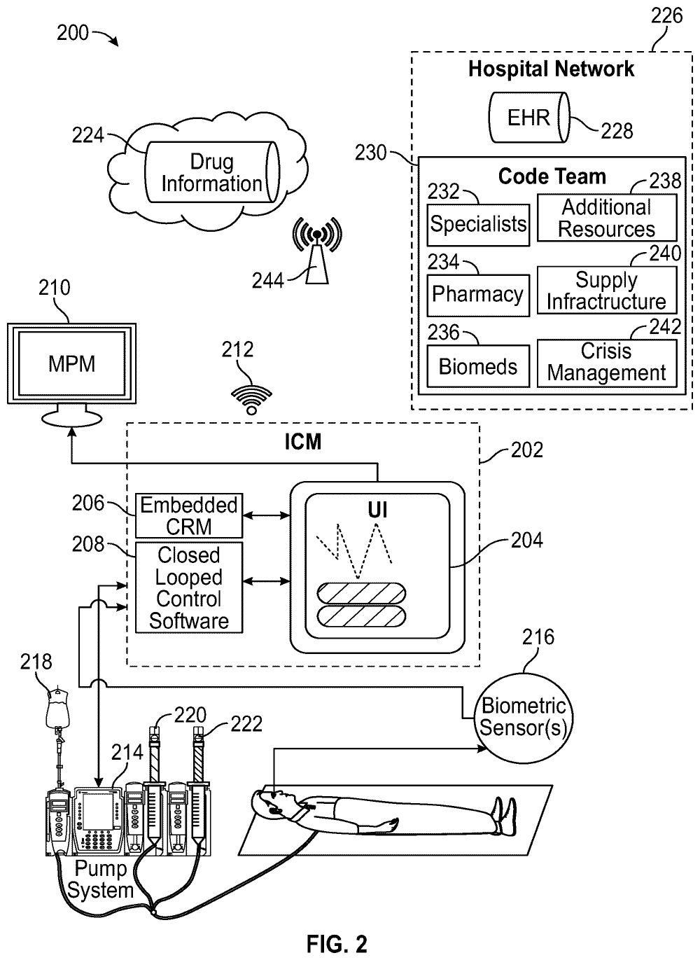

FIG. 2 is a conceptual diagram illustrating an example system architecture, including an example crew resource management unit, according to aspects of the subject technology. In the depicted example, a care provision system 200 includes an intelligent control module (ICM) 202. The ICM 202 provides direct control over connected medical devices to assist the clinician in providing a more centralized control and management of the devices. The ICM 202 provides a modular interface system by which various biometric sensors 216 used in a medical environment may be connected in a generic way to facilitate control over the connected medical devices. For example, the biometric sensors 216 may include one or more of a heart rate monitor, an oxygen sensor, and an intravenous (IV) flow rate monitor, all of which may be connected to the ICM 202 to facilitate, in addition to input by the clinician, centralized control of one or more infusion devices. The ICM 202 may further connect to a server and/or cloud-based system for further data input, data coordination, and reporting. Examples of cloud-based systems include a cloud-based drug information database 224 (or formulary), and a hospital network 226 that includes electronic medical record (EMR) system or database 228.

The hospital network 226 may also include a code team 230 system and/or network that responds to code alerts. The code team 230 includes specialists 232 (human or AI), a pharmacy 234, biomedical technicians 236, crisis management resources 242, supply infrastructure 240, and additional resources 238 for responding to requests made to the code team 230.

In the depicted example, the ICM 202 includes a control unit 208 that provides processing for control algorithms, and connectivity circuitry and/or software to enable closed and semi-closed-loop control capabilities over one or medical devices at a point of use. In some implementations, the ICM 202 provides an external interface, for example a user interface 204, for interaction between an IV infusion pump 214 and one or more different physiological or biometric sensors 216, and for providing input parameters that may be used to control the titration of IV infusions of medications to a patient. For example, syringe pumps 220 and 222 may contain medications (sourced from an IV infusion bag 218) that may be titrated and provided to a patient. In this regard, the ICM 202 can incorporate control software (including, e.g., one or more algorithms in the unit 208) that can be tailored to specific or general medical treatments.

A closed-loop control system as described herein generally refers to a system that does not rely on external manual inputs to deliver a therapy. Once configured, the closed-loop system can autonomously provide a therapy, receive feedback from one or more sensors 216 and, based on the feedback, automatically adjust the therapy as needed. A therapy profile associated with a patient and one or more medical devices involved in the therapy may be generated and/or updated based on the adjustments made by the closed-loop system. A semi-closed-loop control system as described herein is similar to a closed-loop control system except that, in some circumstances, the adjustment to the therapy may depend on an external input. In some implementations, a semi-closed-loop control system may be referred to as a decision support system. Similar to a closed loop system, a therapy profile associated with a patient and/or one or more medical devices involved in the therapy may be generated and/or updated based on the adjustments made by the semi-closed-loop control system.

Having the ICM as a separate unit from the medical device provides several advantages. For example, the advancement of sensors (e.g., biometric sensor(s) 216) may be much more rapid than the development of infusion pump systems (e.g., pump system 214), and thus the control system may accommodate these changes more quickly. It may be desirable to update control algorithms to address changes in treatment methods, available medications, and patient physiology. For this reason, it may be desirable to have the algorithm reside in a component different than the pump system, in order to accommodate more frequent changes. Moreover, machine learning and artificial intelligence may account for patient variations related to physiological parameters such as age, genetics, health history, and other characteristic and environmental factors. Systems incorporating such capabilities may involve large databases and complex programs requiring powerful microprocessors and data storage capabilities to perform the timely and accurate computation needed. These systems are generally not capable of running on the systems currently available with the IV pumps alone, but may reside on a server 30 or other cloud-based system accessible from the ICM.

Additionally, the ICM 202 of the subject technology may be adaptable to a variety of pump systems and sensor inputs. The ICM 202 may further be configured to add wireless, Bluetooth and LAN connections to pump systems that do not currently have it available. For example, the FIG. 2 shows the ICM 202 having a wireless connection 212 for communication with a network system 244 at the hospital. Adding such communications to the pump system 214 may enable other capabilities such as remote monitoring and control of the infusion pumps, and facilitating access to EHR 228. Accordingly, by integrating the electrical and processing components separate from the pump, the subject technology facilitates integration of additional capabilities without needing to modify the pump's housing and electronics. Separating the physiological sensing and control systems from the infusion pump system may further provide for a more streamlined regulatory approval process.

According to various implementations, the ICM 202 facilitates the control software to be separate from the embedded firmware of the pumps and sensors, which can facilitate a scalable and rapidly configured system to provide closed-loop control of medical treatments. Moreover, the ICM 202 can be configured to operate with a multitude of sensors by way of electrical connectors and/or wireless communication. The ICM 202 may include one or more microprocessors and algorithms to provide signal conditioning and/or conversion of the sensor signals to the appropriate physiological parameters for the connected medical device. The parameters may then be used in control algorithms to provide control to, for example, an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

The user interface 204 of ICM 202 can include a display module or a touch-sensitive display module configured to provide a user interface for display of information pertaining to patient physiological status, as well as system control status. In some implementations, the user interface 204 includes circuitry within the ICM 202 housing that provides display information to an external display device. An example of such an external display device includes a multi-parameter monitor (MPM) 210. The MPM 210 may, for example, display various vital statistics (e.g., electrocardiogram saturation level (SpO2)) of a patient in a surgery room such that the vital statistics are visible to the clinicians (e.g., all the clinicians) in the room. The display on the ICM 202 may be mirrored via an associated MPM 210 to provide information to devices connected to the MPM 210 and/or clinicians involved in the treatment.

The ability of the ICM 202 to connect to another display (e.g., the MPM 210) provides modular scalability. For example, a more extensive user interface may be beneficial in some use cases. The more extensive user interface may include displays of data and graphs, for which a larger high-resolution display may be used. Some use cases may benefit from a display that have minimal information and a corresponding user interface to support such a configuration. A smaller, space saving and/or lower cost locally connected display may then be used.

According to various implementations, the ICM 202 includes a crew resource management unit (CRM) 206. The CRM 206 stores protocol information (e.g., information about processes and steps associated with therapies and patients), making the information readily accessible in the ICM 202. In some implementations, the CRM 206 provides information to the user interface 204, allowing the system to more efficiently (e.g., using fewer device resources such as memory, power, processing cycles, user interface area, and the like) present and navigate through pertinent information (e.g., using touch inputs on a touch screen). According to variously implementations, the CRM 206 guides the user to the order of steps and information needed at the appropriate time thereby avoiding expenditures of resources on information that is untimely or irrelevant to the currently detected condition.

In some implementations, the ICM 202 may access additional resources through the communication capabilities of the CRM 206. For example, the CRM 206 may access additional resources relating to medication information and dosage calculations from an internal or remote storage (e.g., from the drug information database 224, from a centralized). In some implementations, the CRM 206 can cause the user interface 204, and/or other operably connected user devices to display a dosage calculator for a patient when a particular medication is to be administered. In some implementations, the dosage calculator is a weight-based calculator that accounts for a weight of the patient (e.g., the weight of the patient is provided to the ICM 202 by manual entry by a clinician, or based on information received from the EHR database 228). In some implementations, calculations for weight-based medications can be readily accessed through the user interface 204 or the one or more user devices communicatively connected to the ICM 202. A patient's electronic health record may also be readily accessed directly through the ICM 202 to provide critical health and allergy information to the clinicians. The additional resources relating to medication information accessed by the ICM 202 via the CRM 206 may include information about a compatibility of medications that a patient is prescribed to receive. In some implementations, the CRM 206 can cause the user interface 204, and/or other user devices to display mixing ratio instructions. For example, the CRM 206 may cause the user interface 204 to specify a current or programmed flow rate of the first fluid from the syringe pump 220, a current or programmed flow rate of the second fluid from the syringe pump 222, and a current or programmed flow rate of the infusion fluid from the infusion bag 218 to facilitate an appropriate mixture for administration to the patient during the infusion.

In one example, the CRM 206 may access additional resources by allowing a clinician to electronically deliver or make pharmacy requests via the ICM 202 to the pharmacy 234. The CRM 206 can cause the user interface 204 to display controls that allow a clinician to summon one or more specialists for consultation (e.g., virtually using cameras communicatively connected to the ICM 202 and/or data from the biometric sensors 216; or request for an in-person consultation at the patient's location). In some implementations, the CRM 206 may connect the clinician to an AI. The CRM 206 can cause the user interface 204 to display controls that allow a clinician to send code alerts so that additional resources and personnel (e.g., human or AI) can respond to a medical issue. The CRM 206 can cause the user interface 204 to display controls for equipment requests or for other resources needed by the patient. Thus, in the cases where a hospital code would need to be issued, a clinician can simply use the CRM 206 to bring up checklists that would then present appropriate codes available for specific requests to be sent via the hospital network 244. In some implementations, the user interface may include a control element that, when activated, transmits a control message to a device in communication with the CRM 206 to request a resource.

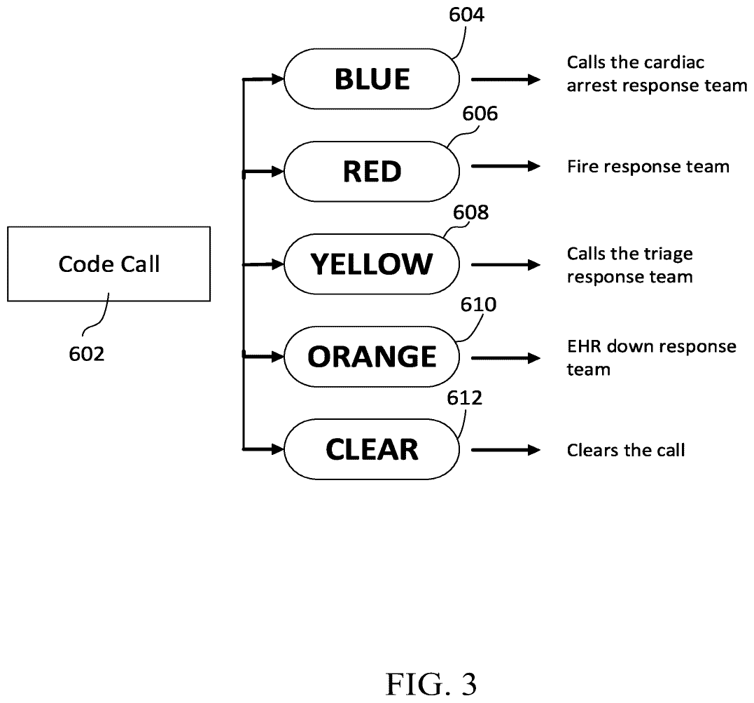

FIG. 3 is a conceptual flow diagram for a code call placed via a user interface control, according to aspects of the subject technology. An example user interface 204 includes a control 602 for triggering a code call. In some implementations, a user input on the control 602 causes a code blue control button 604, a code red control button 606, a code yellow control button 608, a code orange control button 610, and a control button 612 to clear the code call to be displayed in an updated user interface. In some implementations, the control 602 and the code blue control button 604, the code red control button 606, the code yellow control button 608, the code orange control button 610, and the control button 612 to clear the code call are displayed concurrently.

In some implementations, upon receiving a user input on the code blue code button 604, the ICM 202, through a wireless connection 212 with the network system 244 at the hospital, accesses the code team 230 in the hospital network 226. According to various inputs the CRM 206 may make decisions based on user input and/or trained artificial intelligence regarding whether code calls should be sent to the appropriate teams within the hospital organization. For example, CRM 206 may direct the blue code request to a cardiac arrest response team. Accordingly, a code call may be determined and sent automatically, or may be suggested to a clinician for confirmation. When user interaction is indicated the user interface 204 may display one or more code buttons 604-612 to facilitate user action with respect to the code call.

In some implementations, upon receiving a user input on the code red code button 606, the CRM 206 directs, through the wireless connection 212 with the network system 244 at the hospital, the red code request to a fire response team within the code team 230 in the hospital network 226. In some implementations, upon receiving a user input on the code yellow code button 608, the CRM 206 directs, through the wireless connection 212 with the network system 244 at the hospital, the yellow code request to a triage response team within the code team 230 in the hospital network 226. In some implementations, upon receiving a user input on the code orange code button 608, the CRM 206 directs, through the wireless connection 212 with the network system 244 at the hospital, the orange code request to an electronic health records (EHR) down response team. Such a team is triggered when the EHR database 228 malfunctions and/or is inaccessible. When a clinician accidentally sends a code request when none is needed, a control button 612 allows the CRM 206 to clear the call, and/or to communicate with the code team 230 that the code request call is to be cleared.

The CRM 206 may include or be operably connected to decision making algorithms for determining whether a code call should be made or suggested. These algorithms may be in the form of a neural network that is centrally located within the hospital organization's network 10, thereby being in communication with the CRM 206 as well as medical devices 12 and other systems on the network. Such algorithms may be updated, for example, by download of updated software to the ICM 202, or to a server 30 responsible for execution of at least a portion of the algorithms. In this manner, the CRM decision making capabilities may be updated independent of the devices that it monitors and/or controls. According to various implementations, new CRM procedures or changes can be implemented in a more timely and efficient manner by software updates performed over the institution's network (e.g., the network 244).

The ICM 202 includes one or more microprocessors to facilitate execution of the closed loop algorithm, control the infusion pumps, receive the biometric sensor inputs, and provide the user interface (UI) 204 which includes a touch screen display. The user interface enables the entering of patient and procedure information upon which the control loop operates to control the infusion pumps and receive the sensor inputs. The CRM 206 may be part of the firmware that can be called up, manually or automatically in response to an event, through the user interface 204 when an emergency occurs. In addition to the CRM 206, there may be a set-up function that provides a checklist of all the connected systems to ensure they are operational prior to starting the closed loop control.

In cases of emergency, the CRM 206 is used to provide information for handling various situations. In some implementations, the CRM 206 is called up by a clinician (e.g., manually by navigating through the user interface 204, or navigating through a user interface on a user device of the clinician that is communicatively connected to the CRM 206). In some implementations, the protocol information from the CRM 206 is automatically provided to a clinician without requiring user input (e.g., automatically displayed on the user interface 204, when an emergency condition is detected).

According to various implementations, the CRM 206 may provide dynamic variable substitution within the context of a closed loop controlled therapy. In this manner, the CRM may review sensor data and adjustments made to medical devices connected to the ICM 202, and determine whether variables associated with managing control of the medical devices should be substituted and/or changed. In some implementations, variables may be presented to clinicians during the therapy via a display device (e.g., MPM 210 or a display screen associated with the clinician) and input received via the display device or associated input from the clinician.

During the therapy (e.g., during a surgery), messages (e.g., notifications or “tool tips”) may be provided to the clinician(s) based on the stage of the closed loop algorithm at the right time. For example, cardiac related notifications may be presented to a clinician responsible for cardiac therapy (e.g., logged in to the ICM/CRM and associated with the therapy) and anesthesia notifications may be presented to an anesthesiologist responsible for anesthesia (e.g., logged in to the ICM/CRM and associated with the therapy). The therapy may include, for example, surgery within a surgical care area, intubation within an intensive care unit, cardiac resuscitation within an emergency room, or physical therapy in a general hospital or clinical ward.

In some implementations, depending on the procedures, the CRM 206 may attempt to correct the problem by dynamically adjusting operational parameters of the respective medical device and/or the closed loop algorithm before displaying the message or alert to the clinician. In one example in which the CRM detects a loss of sensor signal, the CRM may attempt to reconnect the sensor (e.g., biometric sensor 216) or other device (e.g., display system) by reinitializing or rebooting the circuitry associated with communication with the sensor or device and, if that fails make recoverable recommendations to the clinician.

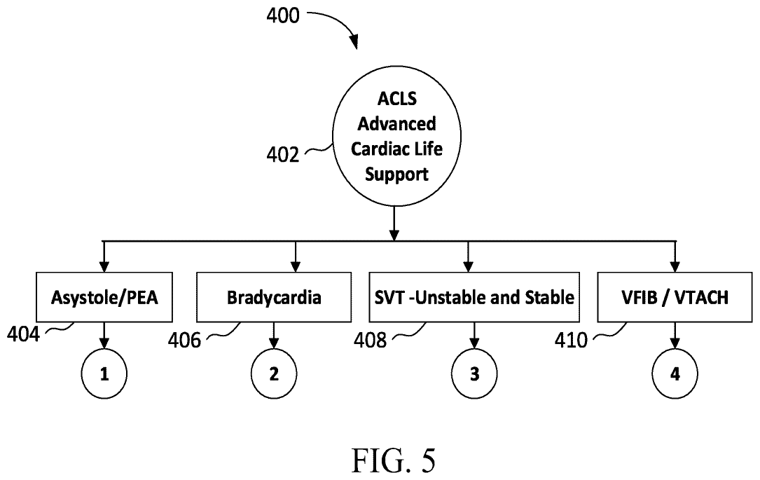

In some implementations, different control signals may be sent to different devices based on different criteria, and at different times. For example, the CRM may manage devices associated with a therapy procedure. The CRM may be preprogrammed with a workflow for the procedure and may be aware of the clinician(s) and device(s) involved in the performance of the procedure or associated with the procedure. The CRM will receive dynamic parameters coming in based on that workflow in addition to monitoring the closed loop system. The CRM may then provide different forms of that information to each clinician according to the clinician's role in the procedure. For example, an anesthesiologist may only receive information required to make decisions about anesthesia (e.g., bispectral sensor information pertaining to the hypnotic state of the patient) and a cardiologist may only receive information required to make cardiology decisions (e.g., cardiac output). In the same manner, each clinician may only receive suggestions and prompts for input that pertain to their specialty in the procedure. Moreover, the algorithm of the CRM may further provide messages at different times to coordinate adjustments in medication and other therapy decisions for the procedure to maintain coordination between the clinicians involved in accordance with a best practice of the healthcare organization. Whether the protocol information provided by the CRM 206 is manually called up by a clinician or automatically provided by the CRM 206, the CRM 206 may additionally provide a user interface that includes a selection panel, as shown in FIG. 5.

FIG. 5 depicts an example selection panel, according to aspects of the subject technology. Turning to FIG. 5, the CRM 206 may cause display of a selection panel 400, based on a hierarchy of emergency conditions. In some implementations, for the most critical emergency condition, Advanced Cardiac Life Support (ACLS) (402) is provided. The display of an indication on the selection panel 400 for ACLS (402) may be automatically provided by the CRM 206 in response to detecting an adverse event pertaining to the patient (e.g., from signals derived from the biometric sensors 216, from other medical devices connected to the patient). Alternatively, the display of an indication on the selection panel 400 for ACLS (402) may be provided in response to a clinician navigating on the user interface 204, or a user interface provided on a user device of the clinician (e.g., the clinician detecting that an adverse event pertaining to the patient is occurring, and seeks further guidance from the ICM 202 for responding to the adverse event in a timely manner). In some implementations, the ACLS relates to the treatment of four conditions: aystole/Pulseless electrical activity (PEA) (404); bradycardia (406); Supraventricular tachycardia (SVT) (408); and ventricular tachycardia, or ventricular fibrillation (410).

In some implementations, the clinician selects an indicator (e.g., “1”) associated with aystole/Pulseless electrical activity (PEA) (404) to cause the CRM 206 to provide protocol information about treating the asystole/PEA condition. Alternatively, when the clinician selects an indicator (e.g., “2”) associated with bradycardia (406), the CRM 206 causes provide protocol information about treating the bradycardia condition to be presented to the clinician(s). When the clinician selects an indicator (e.g., “3”) associated with supraventricular tachycardia (408), the CRM 206 causes provide protocol information about treating both unstable and stable supraventricular tachycardia to be presented to the clinician(s). When the clinician selects an indicator (e.g., “4”) associated with ventricular tachycardia, or ventricular fibrillation (410), the CRM 206 causes provide protocol information about treating ventricular tachycardia or ventricular fibrillation to be presented to the clinician(s).

In some implementations, after the clinician selects from the group of four conditions to treat (e.g., by selecting the indicator corresponding to the condition), the CRM 206 cause a display of recommended steps for treating the selected condition. The display of recommended steps for treating the selected condition includes an order the steps are to be performed in. In some implementations, possible alternative steps are also displayed. In some implementations, the tasks to be performed by each member of the group, and resources for performing the steps are also displayed.

As previously described, the CRM 206, operating under control of AI, may analyze sensor data and make at least one of the foregoing selections on behalf of the clinician. If a hard decision cannot be made (e.g., certain thresholds not being met) then the CRM 206 may suggest a soft decision to the clinician using the selection panel 400. The clinician may then confirm, reject, or change the selection to treat the patient.

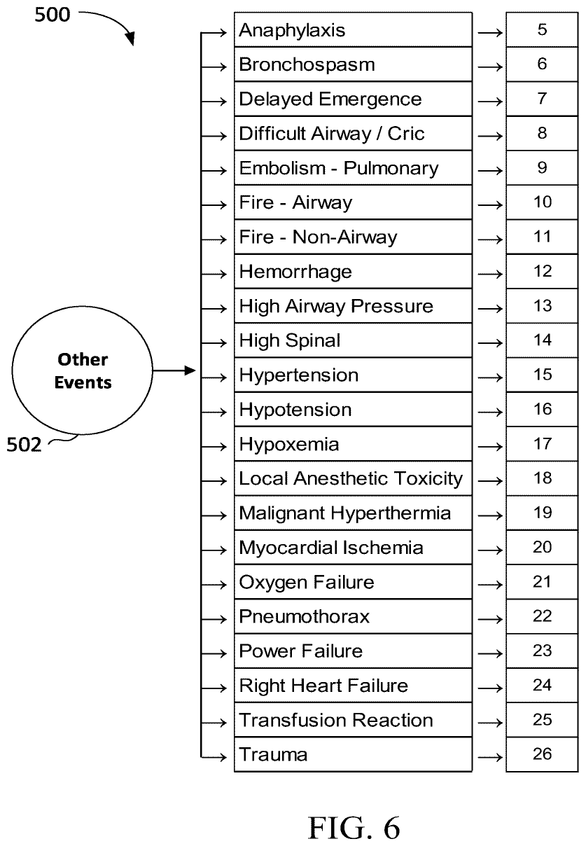

FIG. 6 shows a selection catalog for other emergency conditions, according to aspects of the subject technology. According to the depicted example selection panel 500, the CRM 206 causes display of the example selection panel 500 for emergency conditions that may be less critical than ACLS. As indicated above, the CRM 206 may operate completely under the control of a clinician, or may make decisions independently of the clinician based on analysis of input parameters in the clinical environment (e.g., patient data, sensor data, etc.). In this manner, the decision to display panel 400 and/or the selections made pertaining to panel 400 may be made by the CRM, or suggested to the clinician by the CRM.

The display of an indication on the selection panel 400 for ACLS (402) may be automatically provided by the CRM 206 in response to detecting an adverse event pertaining to the patient (e.g., from signals derived from the biometric sensors 216, from other medical devices connected to the patient). Alternatively, the display of an indication on the selection panel 400 for ACLS (402) may be provided in response to a clinician navigating on the user interface 204, or a user interface provided on a user device of the clinician (e.g., the clinician detecting that an adverse event pertaining to the patient is occurring, and seeks further guidance from the ICM 202 for responding to the adverse event in a timely manner). In some implementations, the ACLS relates to the treatment of four conditions: aystole/Pulseless electrical activity (PEA) (404); bradycardia (406); Supraventricular tachycardia (SVT) (408); and ventricular tachycardia, or ventricular fibrillation (410).

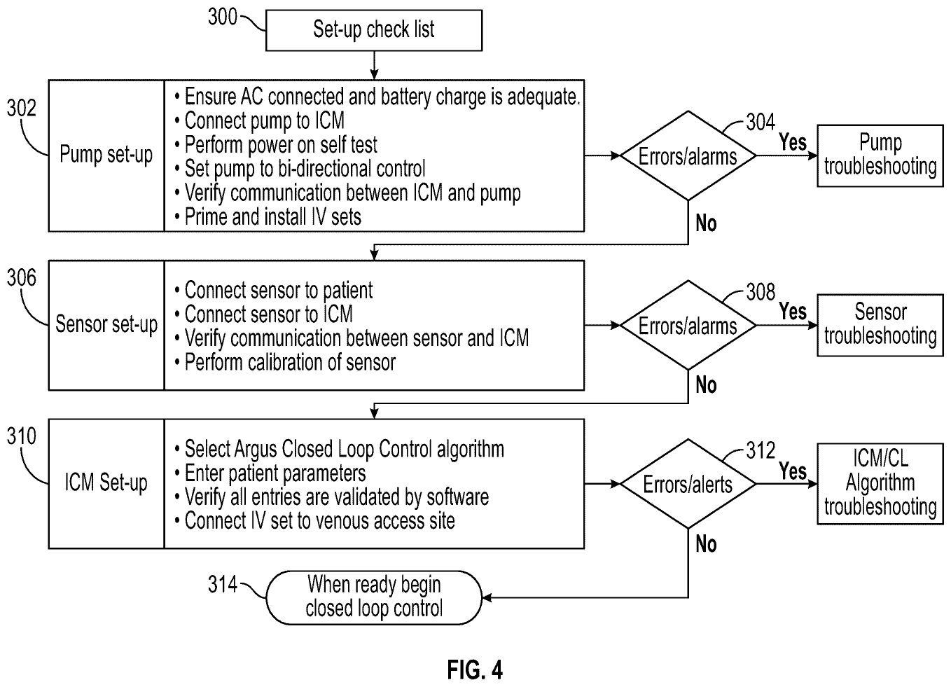

In some implementations, the CRM 206 causes display of a checklist coordination interface (e.g., a checklist coordinator) that allows a clinician (e.g., an anesthetist, a doctor, a nurse) to enter information and verify steps that have been completed (e.g., during a safety check). Verification information may be provided to and/or saved in the CRM 206. Examples of verification information includes, for example, the identity of medical equipment that is provided to the patient (e.g., an infusion pump, a biometric sensor), information about a patient's anesthetic risks (e.g., a value previously computed for the patient, a number of parameters that are used to compute the anesthetic risk), the presence of airway equipment (e.g., oxygen, respirators), the drugs or medications provided or to be provided to the patient, medical devices to be provided to the patient, emergency medications, equipment, and assistance that may be provided to the patient and confirmation about the availability of the equipment, assistance and medications, and confirmation that the equipment is functioning. In some implementations, a therapy profile associated with a patient is built based at least in part on the verification information provided to and/or saved in the CRM 206. During a set-up process for the ICM 202, as described in more detail below, in reference to FIG. 4, adjustments identified and made to one or more medical devices are also recorded and logged by the ICM 202 for further building and updating the therapy profile associated with the patient.

FIG. 4 depicts an example process executed prior to beginning closed loop control, according to aspects of the subject technology. According to the depicted example set-up check list process 300, the CRM 206 may cause display of protocol information for pump set-up (302). In some implementations, the CRM 206 causes the user interface 204 to display check lists during a set-up process. Such check lists provide intelligible and ordered lists of steps to be performed. Further, additional screens can be displayed to provide assistance for troubleshooting, and additional information may be displayed as needed. In some implementations, software in the ICM 202 provides self-checks and verification that the equipment is connected and operating properly prior to starting a procedure. For example, the set-up checklist 300 may begin with setting up the infusion pump (302). The CRM 206 causes protocol information to be displayed at the user interface 204 and/or displays on other user devices. In some implementations, textual protocol information is displayed (e.g., “Ensure AC connected and battery charge is adequate,” “Connect pump to ICM,” “Perform power on self test,” “Set pump to bi-directional control,” “Verify communication between ICM and pump,” “Prime and install IV sets.”). In some implementations, the ICM 202 conducts steps without further input from the users. For example, the power-on self test is performed without active input from the users. While the steps delineated in the pump set-up 302 are performed, the ICM 202 monitors if an error or alarm is triggered (304). If an error or alarm is detected, the ICM 202 proceeds with pump troubleshooting. In some implementations, pump troubleshooting is conducted automatically using closed-loop control, without further user input. An example of such an automated troubleshooting procedure includes rebooting or resetting a communication interface when no communication is detected between the ICM and the pump, resetting the MPM 210, resetting a biometric sensor 216. In some implementations, pump troubleshooting includes identifying adjustments to one or more medical devices (e.g., setting the pump to bi-directional control, adjusting IV sets connected to the pump). Bi-directional communication technology allows the pump to receive the medication order directly from the EHR database 228 and for the pump to send infusion data (medication, rate, dose, volume) to the patient's EHR infusion record in the EHR database 228.

According to the depicted example, when all steps associated with the pump set-up (302) are completed, the ICM 202 causes display of protocol information for sensor set-up (306) at the user interface 204 and/or displays on other user devices. In some implementations, textual protocol information is displayed (e.g., “Connect sensor to patient,” “Connect sensor to ICM,” “Verify communication between sensor and ICM,” “Perform calibration of sensor.”). In some implementations, the ICM 202 conducts steps without further input from the users. For example, the calibration of the sensor is performed without active input from the users. While the steps delineated in the sensor set-up 306 are performed, the ICM 202 monitors if errors or alarms are triggered (308). If an error or alarm is detected, the ICM 202 proceeds with sensor troubleshooting. In some implementations, sensor troubleshooting is conducted automatically using closed-loop control, without further user input. An example of such an automated troubleshooting procedure includes rebooting or resetting a communication interface when no communication is detected between the sensor and the ICM 202. In some implementations, pump troubleshooting includes identifying adjustments to one or more medical devices (e.g., re-calibrating the sensor when a calibration error is detected in the sensor).

When all steps associated with the sensor set-up (306) are completed, the ICM 202 causes display of protocol information for ICM set-up (310) at the user interface 204 and/or displays on other user devices. In some implementations, textual protocol information is displayed (e.g., “Select Argus Closed Loop Control algorithm,” “Enter patient parameters,” “Verify all entries are validated by software,” “Connect IV set to venous access site.”) In some implementations, the ICM 202 conducts steps without further input from the users. For example, verification that all entries input about patient parameters are validated by software is performed without active input from the users. While the steps delineated in the ICM set-up 310 are performed, the ICM 202 monitors if errors or alarms are triggered (312). If an error or alarm is detected, the ICM 202 proceeds with sensor troubleshooting. In some implementations, ICM and/or closed loop troubleshooting is conducted automatically, without further user input. Once all steps in the set-up checklist 300 are completed, the ICM 202 is ready to begin closed loop control.

In some implementations, one or more of the steps (302, 306, 310) may be implemented apart from other steps, and by one or more different processors or devices. Further for explanatory purposes, the steps of the set-up checklist 300 are described as occurring in serial, or linearly. However, multiple steps of the set-up checklist 300 may occur in parallel. In addition, steps of the set-up checklist 300 need not be performed in the order shown and/or one or more of the steps of the set-up checklist 300 need not be performed.

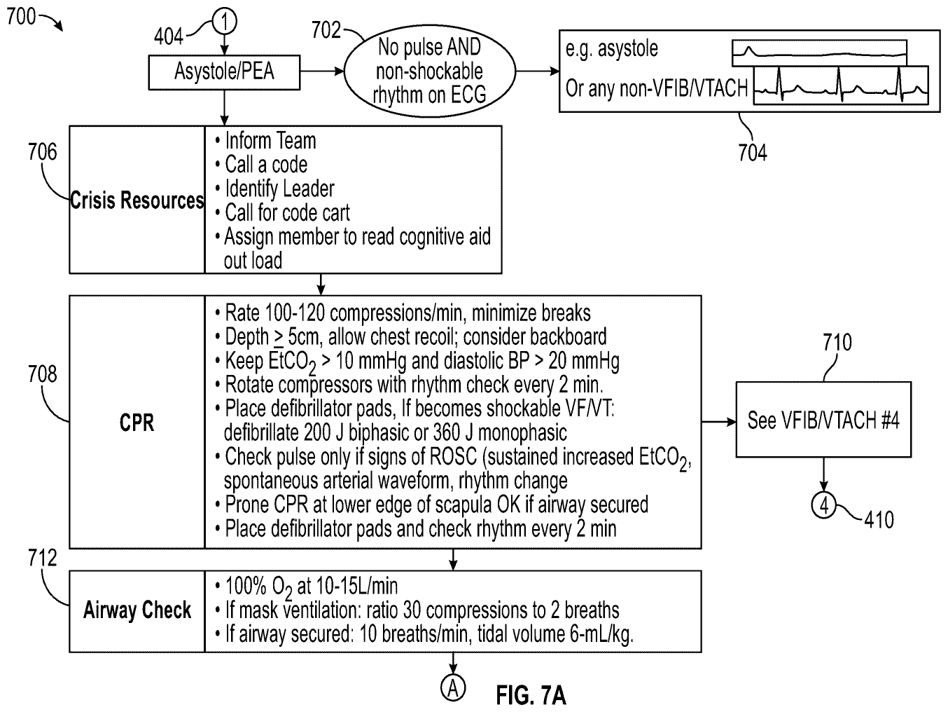

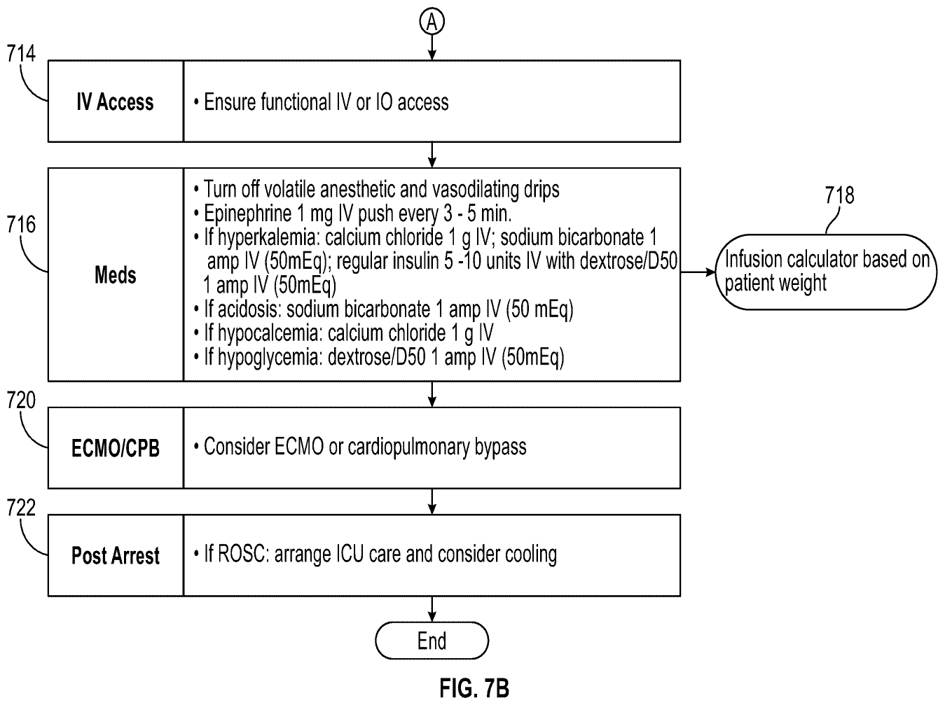

FIG. 7A depicts an example process for intelligently responding to an emergency clinical situation, according to aspects of the subject technology. For explanatory purposes, the various blocks of example process 700 are described herein with reference to FIGS. 7A and 7B, and the components and/or processes described herein. The one or more of the blocks of process 700 may be implemented by a control device such as, for example, one or more computing devices including, for example, ICM 202, or a component thereof. As previously described, the example process 700 may operate under control of AI, which may analyze sensor data and make one or more of the decisions within example process 700 on behalf of the clinician. If a hard decision cannot be made (e.g., certain thresholds not being met) then the CRM 206 may suggest a soft decision to the clinician who may then confirm, reject, or change the selection to treat the patient.

According to the depicted example process 700, in response to the advanced cardiac life support (ACLS) being triggered as registering an asystole (404), the CRM 206 causes a timed sequence of protocol information to be provided by the ICM 202. In some implementations, the triggering of the detection of asystole (404) is caused by user input (e.g., a user selecting the asystole option when presented with the ACLS selection screen). In some implementations, the ICM 202 automatically detects the asystole (404), without requiring user input. For example, the ICM 202 detects signals from the biometric sensors 216 that are indicative of the asystole. The user interface 204 may display explanatory characteristics of asystole (702) in textual form (e.g., “no pulse AND non-shockable rhythm on ECG”) to allow a clinician to confirm that the patient presents a condition that is consistent with asystole. Alternatively, or in addition, the user interface 204 may display sensor information associated with the condition (704). The additional display of textual and sensor information improves safety under potentially stressful emergency situations and helps a clinician confirm the existence of asystole. A user can navigate to the displays of textual explanations or sensor information 704 by interacting directly with the ICM 202, or with a user device (e.g., a tablet, a smart phone, a laptop, a computer) that is in electronic communication with the ICM 202.

The ICM 202 provides crisis resources to one or more clinicians (706). In some implementations, the crisis resources are provided as textual protocol information (e.g., “inform team,” “call a code,” “identify leader,” “call for code cart,” “assignment member to read cognitive aid out loud”). In some implementations, the textual protocol information is provided on the user interface 204 of the ICM 202. In some implementations, the textual protocol information is provided to a user device (e.g., a tablet, a smart phone, a laptop, a computer) of one or more clinicians. In some implementations, the CRM 206 associates specific protocol information with corresponding clinician(s), and selectively provide the protocol information to a respective clinician. For example, instead of displaying the textual protocol information of “identify leader,” the CRM 206 automatically sends a message identifying, as the leader, the most senior clinician in the team of clinicians handling the medical emergency. The CRM 206, can thus provide both decision-making information, and team management guidance, after the emergency condition is triggered. As another example, the CRM 206 can automatically call a code and direct the code to the relevant parties in the care setting, based on input from the biometric sensors 216, without further input from the clinicians.

After all the tasks associated with the crisis resources have been completed, the CRM 206 causes display of protocol information for performing CPR (708). For example, the protocol information includes specific numeral ranges for various procedures or maneuvers (e.g., “rate 100-120 compressions/min, minimize breaks,” “Depth≥5 cm, allow chest recoil; consider backboard,” “Keep EtCO2>10 mmHg and diastolic BP>20 mmHg,” “Rotate compressors with rhythm check every 2 min,” “place defibrillator pads, If becomes shockable VF/VT: defibrillate 200 J biphasic or 360 J monophasic,” “check pulse only if signs of ROSC (sustained increased EtCO2, spontaneous arterial waveform, rhythm change,” “Prone CPR at lower edge of scapula OK if airway secured,” “Place defibrillator pads and check rhythm every 2 min”). In some implementations, tasks associated with the protocol information are automatically assigned to respective members of the team of clinicians, allowing a quicker and more efficient completion of the tasks. In some implementations, the protocol information presented by the ICM 202 is recorded. In some implementations, the protocol information presented by the ICM 202 is recorded and made available for subsequent verification. For example, protocol information that is displayed on the user interface 204 is recorded by the ICM 202, which allows clinicians to analyze, check or verify that processes and procedures were properly carried out according to the protocol information. In some implementations, protocol information that is displayed on user devices of respective clinicians is recorded locally at the user devices or recorded and saved in a cloud storage server associated with the hospital network 244, enabling later retrieval of the presented protocol information. In some implementations, in addition to recording the protocol information presented to clinicians, or instead of recording the presented protocol information, information of actual steps performed by the clinicians is recorded by the ICM 202. For example, the amount of a medication that is provided to a patient, the code requests sent to the code team 230 from the ICM 202, the types and number of medical devices that are operably connected to the ICM 202 (e.g., biometric sensors 216) and the information recorded by the medical devices.

The ICM 202 may determine after one or more steps for the CPR procedure (708) is performed that the patient's medical condition has changed to a different ACLS condition (710). For example, the ICM 202 may determine, based on (updated) input that the patient's condition has changed to VFIB/VTACH (710), which has its own associated set of protocol information. The ICM 202 will then cause protocol information of VFIB/VTACH to be provided to the clinicians in a seamless manner.

According to the depicted example, after all the tasks associated with CPR (708) have been completed, the CRM 206 causes display of protocol information for performing an airway check (712). For example, the protocol information includes specific numeral ranges for various procedures or maneuvers (e.g., “100% O2 at 10-15 L/min,” “If mask ventilation: ratio 30 compressions to 2 breaths,” “If airway secured: 10 breaths/min, tidal volume 6-mL/kg.”) In some implementations, tasks associated with the protocol information are automatically assigned to respective members of the team of clinicians.

FIG. 7B depicts a subsequent portion of the example process shown in FIG. 7A, according to aspects of the subject technology. After all the tasks associated with the airway check (712) have been completed, the CRM 206 causes display of protocol information for IV access (714). For example, the protocol information includes textual protocol information (e.g., “ensure function IV or IO access”).

After all the tasks associated with the IV access (714) have been completed, the CRM 206 causes display of protocol information for medications (716). For example, the protocol information includes textual protocol information (e.g., “Turn off volatile anesthetic and vasodilating drips,” “Epinephrine 1 mg IV push every 3-5 min,” “If hyperkalemia: calcium chloride 1 g IV; sodium bicarbonate 1 amp IV (50 mEq); regular insulin 5-10 units IV with dextrose/D50 1 amp IV (50 mEq),” “If acidosis: sodium bicarbonate 1 amp IV (50 mEq),” “If hypocalcemia: calcium chloride 1 g IV,” “If hypoglycemia: dextrose/D50 1 amp IV (50 mEq).”) In some implementations, the CRM 206 causes display of an infusion calculator that computes an amount of infusion based on a patient's weight (718).

After all the tasks associated with medications (716) have been completed, the CRM 206 causes display of protocol information for Extracorporeal membrane oxygenation (ECMO)/ cardiopulmonary bypass CPB (720). For example, the protocol information includes textual protocol information (e.g., “consider ECMO or cardiopulmonary bypass”).

After all the tasks associated with EMCO/CPB (720) have been completed, the CRM 206 causes display of protocol information for Post Arrest (722). Return of spontaneous circulation (ROSC) is the resumption of a sustained heart rhythm that perfuses the body after cardiac arrest. For example, the protocol information includes textual protocol information (e.g., “If ROSC: arrange ICU care and consider cooling.”). In some implementations, the process 700 terminates after protocol information for Post Arrest (722) has been displayed.

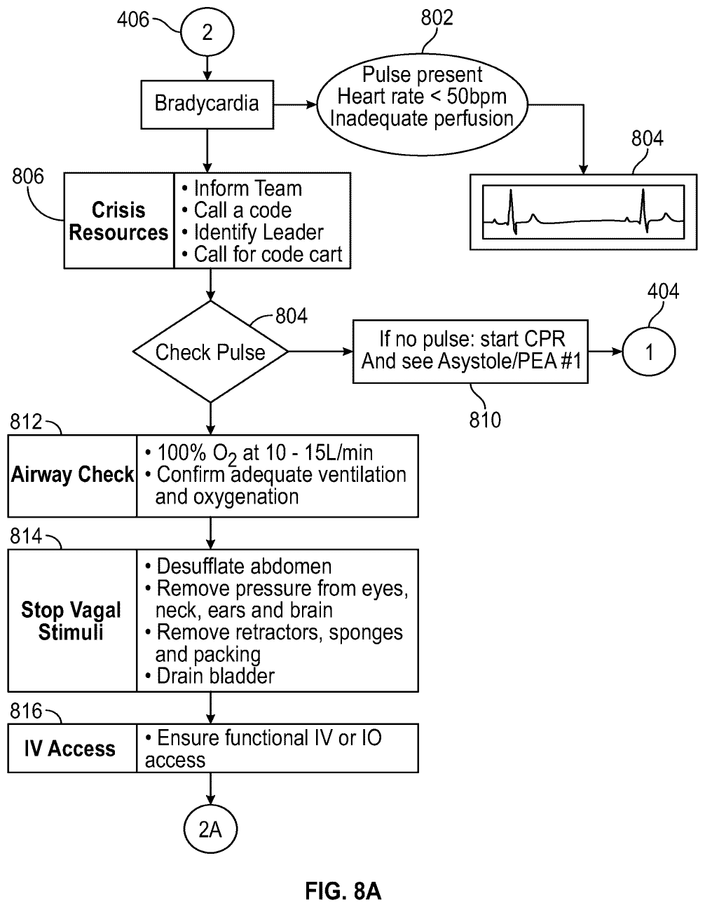

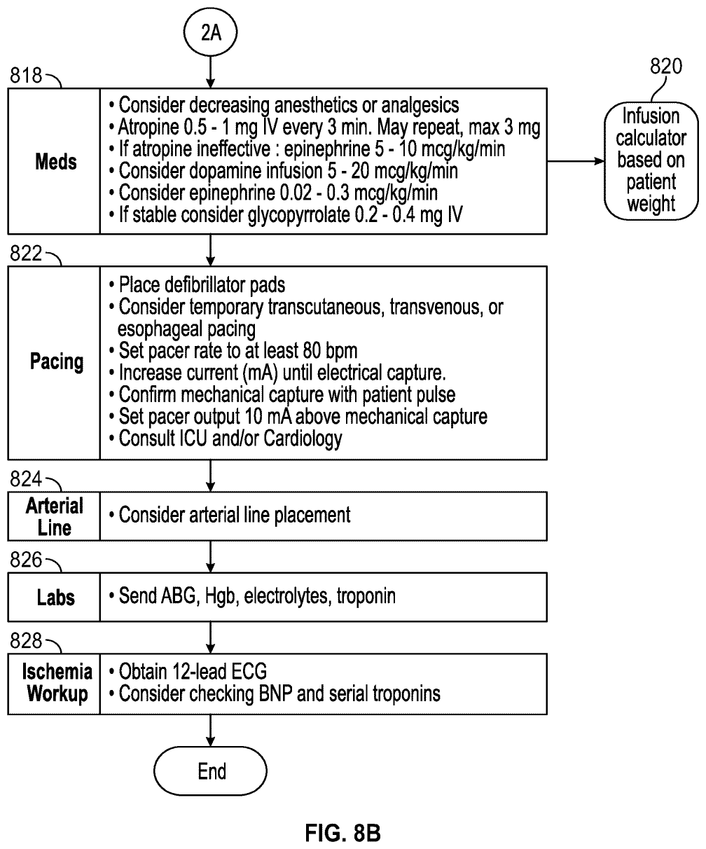

FIG. 8A depicts an example process for intelligently responding to an emergency clinical situation, according to aspects of the subject technology. As described previously, the various blocks of process 700 may be implemented by ICM 202, an associated computing device, or a component thereof, and may further operate under control of algorithms which may make decisions and/or suggest actions to the clinician.

According to the depicted example process 800, when the advanced cardiac life support (ACLS) is triggered as registering a bradycardia (406), the CRM 206 causes a timed sequence of protocol information is provided by the ICM 202. In some implementations, the triggering of the detection of bradycardia (406) is caused by user input (e.g., a user selecting the bradycardia option when presented with the ACLS selection screen). In some implementations, the ICM 202 automatically detects the bradycardia (406), without requiring user input. For example, the ICM 202 detects signals from the biometric sensors 216 that are indicative of the bradycardia. The user interface 204 may display explanatory characteristics of bradycardia (802) in textual form (e.g., “pulse present, heart rate<50 bpm, inadequate perfusion”), or the user interface 204 may display sensor information associated with the condition (804). The additional display of textual and sensor information helps a clinician confirm the existence of bradycardia. A user can navigate to the displays of textual explanations or sensor information 804 by interacting directly with the ICM 202, or with a user device (e.g., a tablet, a smart phone, a laptop, a computer) that is in electronic communication with the ICM 202.

The ICM 202 provides crisis resources to one or more clinicians (806). In some implementations, the crisis resources are provided as textual protocol information (e.g., “inform team,” “call a code,” “identify leader,” “call for code cart.”). In some implementations, the textual protocol information is provided on the user interface 204 of the ICM 202. In some implementations, the textual protocol information is provided to a user device (e.g., a tablet, a smart phone, a laptop, a computer) of one or more clinicians. In some implementations, the CRM 206 associates specific protocol information with corresponding clinician(s), and selectively provide the protocol information to a respective clinician. For example, instead of displaying the textual protocol information of “identify leader,” the CRM 206 automatically sends a message identifying, as the leader, the most senior clinician in the team of clinicians handling the medical emergency. The CRM 206, can thus provide both decision-making information, and team management guidance, after the emergency condition is triggered. As another example, the CRM 206 can automatically call a code and direct the code to the relevant parties in the care setting, based on input from the biometric sensors 216, without further input from the clinicians.

After all the tasks associated with the crisis resources have been completed, the CRM 206 causes display of protocol information for checking a pulse of the patient (808). In response to a determination that there is no pulse, protocol information for starting CPR is displayed. In some implementations, the CRM 206 transitions to displaying information for the asystole (404) conditions automatically, without requiring inputs from the clinician(s).

In response to a determination that there is a pulse, protocol information for airway check (812) is displayed. For example, the protocol information includes specific numeral ranges for various procedures or maneuvers (e.g., “100% O2 at 10-15 L/min,” “Confirm adequate ventilation and oxygenation.”) In some implementations, tasks associated with the protocol information are automatically assigned to respective members of the team of clinicians, allowing tasks to be completed more quickly and/or more efficiently. In some implementations, the protocol information presented by the ICM 202 is recorded and made available for subsequent verification. For example, protocol information that is displayed on the user interface 204 is recorded by the ICM 202, which allows clinicians to analyze, check or verify that processes and procedures were properly carried out according to the protocol information. In some implementations, protocol information that is displayed on user devices of respective clinicians is recorded locally at the user devices or recorded and saved in a cloud storage server associated with the hospital network 244, enabling later retrieval of the presented protocol information. In some implementations, in addition to recording the protocol information presented to clinicians, or instead of recording the presented protocol information, information of actual steps performed by the clinicians is recorded by the ICM 202. For example, the amount of a medication that is provided to a patient, the code requests sent to the code team 230 from the ICM 202, the types and number of medical devices that are operably connected to the ICM 202 (e.g., biometric sensors 216) and the information recorded by the medical devices.

After all the tasks associated with the airway check (812) have been completed, the CRM 206 causes display of protocol information for stopping vagal stimuli (814). For example, the protocol information includes textual protocol information (e.g., “Desufflate abdomen,” “Remove pressure from eyes, neck, ears and brain,” “Remove retractors, sponges and packing,” “Drain bladder.”) In some implementations, tasks associated with the protocol information are automatically assigned to respective members of the team of clinicians.

After all the tasks associated with stopping vagal stimuli (814) have been completed, the CRM 206 causes display of protocol information for IV access (816). For example, the protocol information includes textual protocol information (e.g., “Ensure functional IV or IO access.”) FIG. 8B depicts a subsequent portion of the example process shown in FIG. 8A, according to aspects of the subject technology. After all the tasks associated with the IV access (816) have been completed, the CRM 206 causes display of protocol information for medications (818). For example, the protocol information includes textual protocol information and/or the protocol information includes specific numeral ranges for various procedures or maneuvers (e.g., “Consider decreasing anesthetics or analgesics,” “Atropine 0.5-1 mg IV every 3 min. May repeat, max 3 mg,” “If atropine ineffective: epinephrine 5-10 mcg/kg/min,” “Consider dopamine infusion 5-20 mcg/kg/min,” “Consider epinephrine 0.02-0.3 mcg/kg/min,” “If stable consider glycopyrrolate 0.2-0.4 mg IV.”) In some implementations, the CRM 206 causes display of an infusion calculator that computes an amount of infusion based on a patient's weight (820).

After all the tasks associated with medications (818) have been completed, the CRM 206 causes display of protocol information for pacing (822). For example, the protocol information includes textual protocol information (e.g., “Place defibrillator pads,” “Consider temporary transcutaneous, transvenous, or esophageal pacing,” “Set pacer rate to at least 80 bpm,” “Increase current (mA) until electrical capture,” “Confirm mechanical capture with patient pulse,” “Set pacer output 10 mA above mechanical capture,” “Consult ICU and/or Cardiology.”).

After all the tasks associated with pacing (822) have been completed, the CRM 206 causes display of protocol information for the Arterial Line (824). For example, the protocol information includes textual protocol information (e.g., “Consider arterial line placement.”).

After all the tasks associated with the Arterial Line (824) have been completed, the CRM 206 causes display of protocol information for Labs (826). For example, the protocol information includes textual protocol information (e.g., “Send ABG, Hgb, electrolytes, troponin.”).

After all the tasks associated with labs (826) have been completed, the CRM 206 causes display of protocol information for ischemia workup (828). For example, the protocol information includes textual protocol information (e.g., “Obtain 12-lead ECG,” “Consider checking BNP and serial troponins.”) In some implementations, the process 800 terminates after protocol information for ischemia workup (828) has been displayed.

In the depicted example, the ICM 202 operably connects to an infusion device 206a (302). For example, the ICM 202 may be operably connected to the infusion device by way of a wireless pairing or by way of a wired connection.

Applications of the ICM 202 to control infusion pumps and/or other medical devices, according to various aspects of the subject technology, may be used for closed-loop treatment of a variety of conditions such as anesthesia, blood conditions, blood transfusions, care or medicinal transitions, chemotherapy, enteral therapy, exfiltration, fluid balance conditions, glycemic conditions, hemodynamic conditions, hydration, infiltration, nutritional care, patient controlled analgesic, patient or fluid temperature condition, or vasopressor ventilation. The CRM 206 may be used for all the above mentioned use cases, and protocol information and/or other instructions are presented to clinicians in a use case specific fashion.

For example, in anesthesia, the ICM 202 may monitor an administration of anesthetics to the patient (e.g., an amount, a flow rate, a time interval for administering anesthetics) while using the biometric sensors 216 to monitor and/or display a patient's blood pressure, blood oxygen levels, heart function, and breathing patterns. Anesthesia may require a predetermined amount of an administration of drugs to achieve the required end points of hypnosis, immobility, and suppression of reflexes during a procedure. Examples of the procedure include, for example, surgery within a surgical care area, intubation within an intensive care unit, cardiac resuscitation within an emergency room, or physical therapy in a general hospital or clinical ward. It may be given as the combination of a hypnotic and an opioid, with the anesthesiologist manually titrating doses or infusion rates of the 2 drugs to provide the best balance. Using a bispectral index sensor (BIS), the ICM 202 may monitor the depth of anesthesia and use closed-loop control of the infusion device to administer a proper amount of an IV drug(s), while preventing awareness or excessive anesthetic depth during medical treatment, thereby improving patients'outcomes. For the anesthetic closed-loop control use case, as well as the other use cases, the CRM 206 enables an anesthesiologist to command and control all resources at hand to execute the anesthesia as planned and respond to a problem that may arise. Such an approach allows transferring of the knowledge of what needs to be done for a patient into an effective team activity. In some implementations, the CRM 206 classifies a process to be carried out into one of two types: information for decision-making elements and team management components which allows more effective problem solving in a complex, ill-structured environment.

As another example, the ICM 202 may be connected to biometric sensor 216 that is configured as a blood glucose monitor and, based on intelligent modeling and continuous blood glucose measurements, maintain a patient's blood glucose by way of controlling an infusion device's administration of insulin and/or dextrose solution(s). Blood glucose (BG) disorders, such as stress-induced hypoglycemia and hyperglycemia, can be common complications in patients in the ICU. In addition, patients with type 1 and 2 diabetes may be susceptible to hyperglycemia, as well as severe hypoglycemia as a result of overcorrection with insulin. For this reason, IV infusions of insulin that are controlled by the ICM 202 using a closed-loop configuration with inputs from continuous BG measurements is an ideal application for IV infusions of inulin and dextrose to maintain BG levels within the desired range.

As yet another example, a vasopressor based therapy can require frequent boluses, and adjustment of infusion rates expediently to avoid harmful periods of hypotension or hypertension. The foregoing implementation may also be used to control sepsis treatment which requires antibiotic administration and fluid resuscitation to correct hypotension. Such IV fluid management is important for sepsis patients and controlling the timing, type, and amount of fluid administered is critical since excess quantities of IV fluids could also be detrimental.

In various implementations, the ICM 202 may receive input based on one or more intelligent models, and the specific user case, and/or protocols and/or simulations to make decisions for controlling an infusion device. Such models, protocols, and simulations may be based, at least in part, on machine learning algorithms that process training data input based on a population of patients having similar conditions to the patient being treated.

In some implementations, multiple simultaneous closed-loop therapies (e.g., IV Glycemic control and Hemodynamic stability) can be hosted by the ICM 202 while running one or more closed-loop algorithms with simultaneous connection to the different sensors and actuators. An alternate implementation could use multiple ICMs each running a single closed-loop therapy.

In some implementations, the ICM 202 obtains (e.g., downloads) a first algorithm from a server based on determining that the one or more sensor devices are operably connected to the ICM 202. The ICM 202 may first confirm that the one or more sensor devices are associated with a first therapy and a type of the infusion device before downloading the first algorithm. The ICM 202 may also download additional algorithms after determining that a new sensor has been operably connected. The first algorithm may be configured to operate the infusion device based on real-time data received from the one or more sensor devices. For example, the first algorithm may be configured to operate the infusion device in a closed-loop mode based on the real-time data received from the one or more sensor devices. The ICM 202 may also receive a configuration parameter associated with operation of the one or more infusion devices.

According to various implementations, the modularity of the system provides for extensibility and a longer lifecycle through dynamic updates. For example, a new sensor may be operably connected to the ICM 202. Upon the ICM 202 detecting the new sensor, the ICM 202 may download a second algorithm associated with the new sensor and update the user interface to display a new data module associated with the new sensor. Data may then be received from the new sensor and displayed via the new data module. The infusion device may then be controlled within the closed-loop mode based on real-time data received from the new sensor and the one or more sensor devices, the downloaded first and second algorithms, and the received configuration parameter.

In various implementations, the ICM 202 builds a therapy profile associated with a patient and the medical devices based on the identified adjustments made to one or more of the medical devices. In some implementations, the identified adjustments are made by the closed-loop control systems, without additional user input.

The ICM 202 may identify an adverse event pertaining the patient or a respective medical device operably connected to the ICM 202. For example, data received from the biometric sensors 216 may indicate that the patient receiving a medical fluid (e.g., anesthetics, insulin or other medications for glycemic control, vasopressor, for infusion fluids), has an abnormal reading for one or more of: blood pressure, blood oxygen level, or heart function, or breathing pattern. Prior to and/or during the therapy (e.g., receiving one or more medical fluids based on the specific use case), the ICM 202 creates and refines a therapy profile associated with the patient. In some implementations, the ICM 202 monitors one or more medical devices operably connected to the ICM 202, and adjusts the medical devices over a period of time during and/or after the therapy.

The ICM 202, in response to identifying the adverse event, may generate, based on the therapy profile, on or more consecutive control signals to correct the adverse event. In some implementations, the adverse event corresponds to the ACLS events described in FIG. 5, or the events described in FIG. 6. Each of the consecutive control signal is associated with a different one of the medical devices or a different user. In some implementations, the consecutive control signals include a display control signal. For example, the display control signal is a first control signal that causes a first message to be displayed on a first device (e.g., “call a code,” “inform team,” “call for code cart”), and a second control signal of the plurality of control signals causes a second message to be displayed on a second device at a different time than when the first message is displayed on the first device (e.g., “keep EtCo2>10 mmHg and diastolic BP>20 mmHg). The display control signal causes the display of one or more of the messages and protocol information described in FIGS. 7A-8B. In some implementations, the consecutive control signals generate display control signals at user devices of different clinicians (e.g., a user device of an anesthesiologist and a user device of a nurse).

The systems and methods described herein can also provide context-sensitive help and/or checklists during different phases of use of the ICM 202, including the initial setup and operation. For example, a user can be guided in an initial setup of the ICM 202, or medical devices that are to be operably connected to the ICM 202 using a web or an app interface. In some implementations, prior to installing and/or turning on of the ICM 202, an initial setup guide may be provided on one or more medical devices and or web screen that is used by a technician or a staff member in charge of setting up the medical devices and/or ICM 202.

In some implementations, the installation includes assembling a kit for a particular use case (e.g., blood glucose detectors for glycemic control, biometric sensors for vasopressor therapy). The kit may include identification tags such as barcodes, AprilTags (e.g., a visual fiducial system useful for a wide variety of tasks including augmented reality, robotics, and camera calibration), near-field communication (NFC) tags, non-fungible tokens (NFT). In some implementations, the identification tags can interact with the ICM 202 (e.g., via NFC communication means, BlueTooth, etc.) When scanned by a user, the identification tag causes relevant instructions for connecting the kit to the ICM 202 to be displayed to the user. In some implementations, the CRM 206 provides a user interface for displaying checklists and/or receiving input from the user for checking off components in an assembled kit (e.g., by recording a serial number, lot number for disposables).

During operation, the CRM 206 is readily accessible on the ICM 202, or a web screen on an external device (e.g., an external tablet, phone, computer). In some implementations, when a clinician is to attend to a component of the therapy that is not electronically paired to the ICM 202 (e.g., disposable set, a disconnected or malfunctioning sensor), the clinician is able to scan the component (e.g., an identification tag on the component) and receive context-sensitive instructions associated with that component. For example, if a disconnected or malfunctioning sensor (e.g., the sensor has bad signal quality) was previously paired and operating, scanning an AprilTag associated with the sensor would now provide instructions for recovering the sensor or replacing the sensor with a new one. In other words, once an identification tag associated with a particular tool is received, the CRM 206 is able to present a tip relating to the tool that is relevant to the user.

In some implementations, when the system (e.g., the algorithm) recognizes the user, for example, through a login or other types of identity management, the system may choose to switch to providing set up instructions using an “expert mode,” and skip the more basic instructions. In some implementations, the system determines which steps have already been completed by the user during the set-up process and jumps to a specific step by skipping over all the previously completed steps. In some implementations, different installation instructions are provided to different users, and the system routes different messages to different people at the right time and in the right sequence, which may significantly shorten the set-up process. For example, a user no longer has to actively keep track of where in the set-up process he is, in order to move on to the next step in the process-the system will automatically provide the necessary information to the user at the right time and in the right sequence. In some implementations, the system also permits real-world modification during the installation process. For example, the system may determine that a medical device has been accidentally unplugged during the installation process, and the system can directly provide instructions to address the error triggered by the disconnection, presenting an instruction to the user at the correct time, instead of troubleshooting the error at a later time point.

In some implementations, a medical device (e.g., biometric sensor 216) may include a microcontroller that communicates with a microcontroller of the ICM 202 to facilitate a secure handshake with the ICM 202 and/or the infusion device. The ICM 202, together with a corresponding infusion device and accessory devices, forms an intelligent accessory system that increases safety by reducing human error and accessory damage. In some implementations, the CRM 206 may include software instructions configured to facilitate automatic setup (e.g., on the infusion device) of infusion parameters related to a connected accessory. In some implementations, each accessory may include software or firmware configured to provide self-diagnosis of hardware/firmware issues which can then be communicated to the infusion device via the ICM 202. The ICM 202 may further communicate with each connected medical device to ensure proper functionality (e.g., via reporting from the medical device) and automatically disable medical devices which do not report a valid status, thereby preventing users from utilizing the wrong or damaged medical device.

According to various implementations, the ICM 202 may function as an Intelligence Device Hub (IDH) having multiple hardware ports, each including a universal connector. All connected medical devices communicate via a common internal bus, alleviating the need for dedicated ports; that is, a variety of medical device may be connected to an I/O port to communicate with the infusion device via a common digital communication protocol. In this regard, new medical devices that implement a common protocol associated with the IDH and which utilize the connector can be implemented without updates to the IDH or the infusion device. Software updates may be pushed to the medical devices via the IDH.

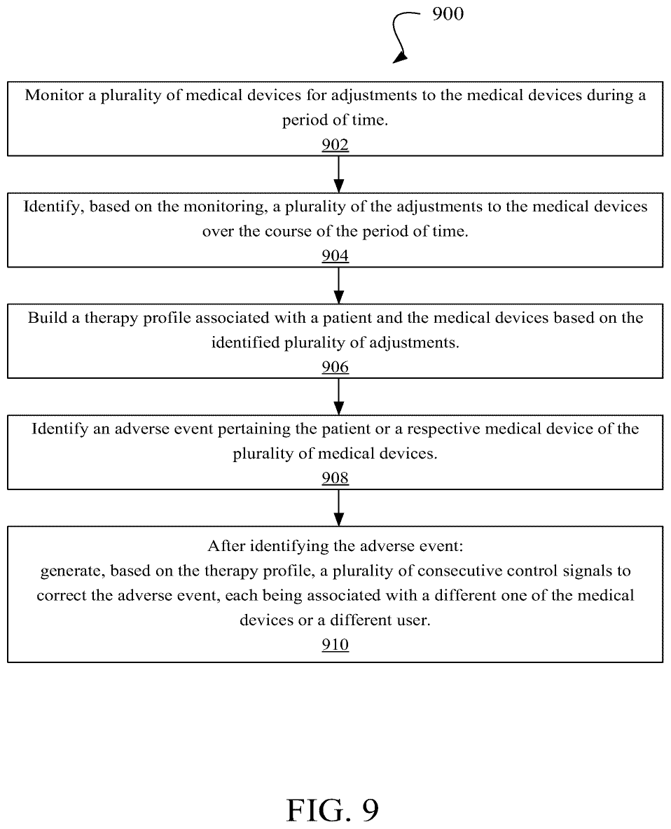

FIG. 9 depicts an example process medical device resource management, according to aspects of the subject technology. For explanatory purposes, the various blocks of example process 900 are described herein with reference to FIGS. 1 through 8B, and the components and/or processes described herein. The one or more of the blocks of process 900 may be implemented, for example, by one or more computing devices including, for example, server 30, ICM 202, or a component thereof. Additionally, or in the alternative, as previously described, the example process 900 may operate under control of AI, which may analyze sensor data and make one or more of the decisions within example process 900 on behalf of the clinician. If a hard decision cannot be made (e.g., certain thresholds not being met) then the CRM 206 may suggest a soft decision to the clinician who may then confirm, reject, or change the selection to treat the patient.

In some implementations, one or more of the blocks may be implemented apart from other blocks, and by one or more different processors or devices. Further for explanatory purposes, the blocks of example process 900 are described as occurring in serial, or linearly. However, multiple blocks of example process 900 may occur in parallel. In addition, the blocks of example process 900 need not be performed in the order shown and/or one or more of the blocks of example process 900 need not be performed.

The system executing the example process 900 includes an ICM 202 that includes the CRM 206, and an infusion device in communication with the ICM 202 via one of the ports.

In the depicted example, the ICM 202 monitors a plurality of medical devices that is operably connected to the ICM 202 for adjustments to the medical devices during a period of time (902).

The ICM 202 identifies, based on the monitoring, one or more adjustments to the medical devices over the course of the period of time (904). In this regard, the ICM 202 may identify and/or log the adjustments to the medical devices during the period of time. The ICM 202 builds a therapy profile associated with a patient and the medical devices based on the identified plurality of adjustments (906). The ICM 202 identifies an adverse event pertaining the patient or a respective medical device of the plurality of medical devices (908). After identifying the adverse event, the ICM 202 (e.g., CRM 206) generates, based on the therapy profile, a plurality of consecutive control signals to correct the adverse event, each being associated with a different one of the medical devices or a different user (910).

In some implementations, the ICM 202 sends each of the plurality of consecutive control signals to a different device associated with a different user. There may be more than one clinician in the clinical environment. For example, there may be one or more doctors, and one or more nurses and/or surgical technicians working together to provide a therapy to a patient (e.g., in an operating room). Accordingly, the CRM 206 may monitor the medical devices that are associated with a procedure being performed during the period of time, and provide suggestions or instructions to each clinician individually. Examples of the procedure include, for example, surgery within a surgical care area, intubation within an intensive care unit, cardiac resuscitation within an emergency room, or physical therapy in a general hospital or clinical ward. In some implementations, each clinician may receive instructions from a different device or display screen, or section of a user interface within a MPM 210. In this regard, the CRM may provide a first control signal (of the consecutive control signals) to a first device, which causes a first message to be displayed on the first device, and a second control signal of the plurality of consecutive control signals causes a second message to be displayed on a second device. The second message may be displayed, for example, at a different time than when the first message is displayed on the first device. In some implementations, the second control signal is sent to a device outside of an area associated with the procedure. When the adverse event includes a sensor failure, the ICM may send one of the consecutive control signals to a medical device. In other words, the control signal includes a message for adjusting one of the medical devices. In some implementations, a first control signal of the plurality of consecutive control signals may include a reset command configured to cause a restart an operation of a medical device.

According to various aspects, the disclosed medical device resource management system 202 includes a communication interface configured to exchange information with a plurality of medical devices, and to exchange information with at least one of a plurality of user devices. The medical devices may include, for example, at least one infusion device 214, and the user devices may include an MPM 210 connected to the disclosed ICM 202 and/or one or more displays or associated devices associated with respective clinicians. In this regard, the system 202 includes a processor and a non-transitory computer readable medium with instructions stored thereon that, when executed by the processor, cause the medical device resource management system to monitor the plurality of medical devices for adjustments to the plurality of medical devices during a period of time. For example, during a medical procedure a closed loop system, or semi closed loop system may make adjustments to the medical device to stabilize the patient during the medical procedure. The clinicians may make further adjustments to medical device(s) during the procedure. In this regard, the ICM 202 identifies, based on the monitoring, the adjustments to the plurality of medical devices during the period of time and builds a therapy profile associated with a patient and the plurality of medical devices based on the identified plurality of adjustments. The ICM may then identify an adverse event pertaining the patient or a respective medical device of the plurality of medical devices. For example, the ICM may determine via one or more biometric sensors that the patient has become unstable. For example, a cardiac monitor 216 may indicate that the patient has entered into an abnormal rhythm or a bispectral sensor 216 may indicate that the patient is no longer in a hypnotic state or is trending towards being awake. After identifying the adverse event, the ICM 202 may generate, based on the therapy profile, a plurality of consecutive control signals to correct the adverse event, each being associated with a different one of the medical devices or a different user. In a closed loop system, a respective control signal may be directed to adjusting an infusion of a vasopressor to stabilize the patient's cardiac output, or adjusting an amount of an anesthesia drug to maintain the patient in the hypnotic state. In a semi-closed loop system, the control signal may be a suggestion sent to a device associated with the clinician responsible for overseeing the respective drug. For example, a notification regarding the cardiac output and adjustment to the vasopressor may be sent to the cardiac surgeon, while the notification regarding the anesthesia drug may be sent to the anesthesiologist.

Accordingly, the medical device resource management system may be configured to send each of the plurality of consecutive control signals to a different device associated with a different user. In some implementations, the monitoring the plurality of medical devices includes monitoring medical devices that are associated with a procedure being performed during the period of time. In some implementations, a first control signal of the plurality of consecutive control signals causes a first message to be displayed on a first device, and a second control signal of the plurality of consecutive control signals causes a second message to be displayed on a second device at a different time than when the first message is displayed on the first device. In some implementations, the second control signal is sent to a device outside of an area associated with the procedure. In some implementations, the adverse event includes a sensor failure. As described previously, the consecutive control signals may include a message for adjusting one of the medical devices.

In some implementations, the medical device resource management system is configured to receive information about roles of respective users associated with corresponding user devices. Such information may be obtained from the user devices or from the hospital information server 30 which, according to various implementations, is responsible for authorization of the users to the user devices and/or the ICM 202. The ICM 202 may cause a display, based on the plurality of consecutive control signals, of different protocol information at the corresponding user devices based on the roles of respective users. In some implementations, the consecutive control signals may cause display of protocol information after receiving a user selection of a therapy (e.g., during setup 310 of the ICM for the procedure). The user selection of the therapy may include a therapy for one of asystole, bradycardia, ventricular tachycardia, or ventricular fibrillation.

The ICM 202 may receive verifications of one or more actions performed in response to the user selection of the therapy, and cause dynamic protocol information to be displayed based on the verifications of the one or more actions. The protocol information may include emergency protocol information, and the emergency protocol information may be provided in accordance with a determination by the ICM 202 that a close-loop control algorithm associated with and controlling the medical devices fails to correct an anomaly detected during the period of time. The ICM 202 may update, delete, or download emergency protocol information via the communication interface.

In another aspect, an example machine-implemented method includes monitoring a plurality of medical devices for adjustments to the medical devices during a period of time, identifying, based on the monitoring, a plurality of the adjustments to the plurality of medical devices during the period of time, building a therapy profile associated with a patient and the medical devices based on the identified adjustments, identifying an adverse event pertaining the patient or a respective medical device of the plurality of medical devices; after identifying the adverse event, generating, based on the therapy profile, a plurality of consecutive control signals to correct the adverse event, each being associated with a different one of the medical devices or a different user. As described previously, the method may further include receiving, from a plurality of user devices, information about roles of respective users associated with corresponding user devices, and displaying, based on the plurality of consecutive control signals, different protocol information at the corresponding user devices based on the roles of respective users.

According to various aspects described herein, the subject technology further includes a non-transitory machine-readable medium storing instructions thereon that, when executed by a device, cause the device to perform a machine-implemented method described above. In some implementations, the device includes the disclosed ICM 202. Additionally, or in the alternative, the device or devices may include an infusion control device.

Many of the above-described example 900, and related features and applications, may also be implemented as special purpose software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, combinations of separate programs that together implement a software aspect described here is within the scope of the subject disclosure. In some implementations, the software programs, when installed to operate on one or more electronic systems, define one or more specific machine implementations that execute and perform the operations of the software programs causing one or more devices to implement crew resource management features described.

A computer program (also known as a program, software, software application, script, or code) can be written in a programming language, including compiled or interpreted languages, declarative or procedural languages, and it can be deployed for execution, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 10 is a conceptual diagram illustrating an example electronic system 1000 for intelligent operation of infusion accessory devices, according to aspects of the subject technology. Electronic system 1000 may be a specially configured computing device for execution of software associated with one or more portions or steps of process 900, or components and processes provided by FIGS. 1-9, including but not limited to information system server 30, database 37, computing hardware within patient care device 12, or a remote device 32 (e.g., a mobile device). Electronic system 1000 may be representative, in combination with the disclosure regarding FIGS. 1-9. In this regard, electronic system 1000 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or other specifically configured computer-related electronic device having network connectivity.

Electronic system 1000 may include computer readable media and interfaces for computer readable media. In the depicted example, electronic system 1000 includes a bus 1008, processing unit(s) 1012, a system memory 1004, a read-only memory (ROM) 1010, a permanent storage device 1002, an input device interface 1014, an output device interface 1006, and one or more network interfaces 1016. In some implementations, electronic system 1000 may include or be integrated with other computing devices or circuitry for operation of the various components and processes previously described.

Bus 1008 collectively represents system, peripheral, and chipset buses that communicatively connect the numerous internal devices of electronic system 1000. For instance, bus 1008 communicatively connects processing unit(s) 1012 with ROM 1010, system memory 1004, and permanent storage device 1002.

From these various memory units, processing unit(s) 1012 retrieves instructions to execute and data to process, in order to execute the processes of the subject disclosure. The processing unit(s) can be a single processor or a multi-core processor in different implementations.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system 1000 is off. Some implementations of the subject disclosure use a mass-storage device (such as a magnetic or optical disk and its corresponding disk drive) as permanent storage device 1002.

Other implementations use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such as a random access memory. System memory 1004 stores some of the instructions and data that the processor needs at runtime. In some implementations, the processes of the subject disclosure are stored in system memory 1004, permanent storage device 1002, and/or ROM 1010. From these various memory units, processing unit(s) 1012 retrieves instructions to execute and data to process in order to execute the processes of some implementations.

Bus 1008 also connects to input and output device interfaces 1014 and 1006. Input device interface 1014 enables the user to communicate information and select commands to the electronic system. Input devices used with input device interface 1014 include, e.g., alphanumeric keyboards and pointing devices (also called “cursor control devices”). Output device interfaces 1006 enables, e.g., the display of images generated by the electronic system 1000. Output devices used with output device interface 1006 include, e.g., printers and display devices, such as cathode ray tubes (CRT) or liquid crystal displays (LCD). Some implementations include devices such as a touchscreen that functions as both input and output devices.

Also, as shown in FIG. 10, bus 1008 also couples electronic system 1000 to a network (not shown) through network interfaces 1016. Network interfaces 1016 may include, e.g., a wireless access point (e.g., Bluetooth or WiFi) or radio circuitry for connecting to a wireless access point. Network interfaces 1016 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 1000 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and the claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and the claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received such as acoustic input, speech input, gesture input, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or a combination of one or more such back end, middleware, or front end components. The components of the system can be interconnected by forms or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as specifically configured electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for medical device resource management, the method comprising, under control of one or more processing devices: monitoring a plurality of medical devices for adjustments to the medical devices during a period of time; identifying, based on the monitoring, the adjustments to the plurality of medical devices during the period of time; building a therapy profile associated with a patient and the medical devices based on the identified adjustments; identifying an adverse event pertaining the patient or a respective medical device of the plurality of medical devices; and after identifying the adverse event: generating, based on the therapy profile, a plurality of consecutive control signals to correct the adverse event, each being associated with a different one of the medical devices or a different user.

Clause 2. The method of Clause 1, further comprising: sending each of the plurality of consecutive control signals to a different device associated with a different user.

Clause 3. The method of Clause 1 or Clause 2, wherein the monitoring the plurality of medical devices comprises monitoring medical devices that are associated with a procedure being performed during the period of time.

Clause 4. The method of Clause 1 or Clause 2, wherein a first control signal of the plurality of consecutive control signals causes a first message to be displayed on a first device, and a second control signal of the plurality of consecutive control signals causes a second message to be displayed on a second device at a different time than when the first message is displayed on the first device.

Clause 5. The method of Clause 4, wherein the second control signal is sent to a device outside of an area associated with a procedure being performed during the period of time.

Clause 6. The method of any one of Clauses 1 through 5, wherein the adverse event comprises a sensor failure, wherein one of the consecutive control signals comprises a message for adjusting one of the medical devices.

Clause 7. The method of any one of Clauses 1 through 5, wherein a first control signal of the plurality of consecutive control signals comprises a reset command configured to cause a restart an operation of a medical device.

Clause 8. A medical device resource management system, comprising: a communication interface configured to exchange information with a plurality of medical devices, and to exchange information with at least one of a plurality of user devices; a processor; and a non-transitory computer readable medium including instructions that, when executed by the processor, cause the medical device resource management system to: monitor the plurality of medical devices for adjustments to the plurality of medical devices during a period of time; identify, based on the monitoring, a plurality of the adjustments to the plurality of medical devices during the period of time; build a therapy profile associated with a patient and the plurality of medical devices based on the identified plurality of adjustments; identify an adverse event pertaining the patient or a respective medical device of the plurality of medical devices; and after identifying the adverse event: generate, based on the therapy profile, a plurality of consecutive control signals to correct the adverse event, each being associated with a different one of the medical devices or a different user.

Clause 9. The medical device resource management system of Clause 8, wherein the medical device resource management system is configured to send each of the plurality of consecutive control signals to a different device associated with a different user.

Clause 10. The medical device resource management system of Clause 8 or Clause 9, wherein monitoring the plurality of medical devices comprises monitoring medical devices that are associated with a procedure being performed during the period of time.

Clause 11. The medical device resource management system of Clause 8 or Clause 9, wherein a first control signal of the plurality of consecutive control signals causes a first message to be displayed on a first device, and a second control signal of the plurality of consecutive control signals causes a second message to be displayed on a second device at a different time than when the first message is displayed on the first device.

Clause 12. The medical device resource management system of Clause 11, wherein the second control signal is sent to a device outside of an area associated with the procedure.

Clause 13. The medical device resource management system of any one of Clauses 8 through 12, wherein the adverse event comprises a sensor failure, wherein one of the plurality of consecutive control signals comprises a message for adjusting one of the medical devices.

Clause 14. The medical device resource management system of any one of Clauses 8 through 13, wherein the medical device resource management system is configured to: receive, from a plurality of user devices, information about roles of respective users associated with corresponding user devices; and display, based on the plurality of consecutive control signals, different protocol information at the corresponding user devices based on the roles of respective users.

Clause 15. The medical device resource management system of any one of Clauses 8 through 14, wherein the plurality of consecutive control signals causes display of protocol information after receiving a user selection of a therapy.

Clauses 16. The medical device resource management system of Clause 15, wherein the user selection of the therapy comprises a therapy for one of asystole, bradycardia, ventricular tachycardia, or ventricular fibrillation.

Clause 17. The medical device resource management system of any one of Clauses 8 through 16 wherein the medical device resource management system is further configured to: receive verifications of one or more actions performed in response to the user selection of the therapy; and cause dynamic protocol information to be displayed based on the verifications of the one or more actions.

Clause 18. The medical device resource management system of Clause 15, wherein the protocol information comprises emergency protocol information, and wherein the emergency protocol information is provided in accordance with a determination by the medical device resource management system that a close-loop control algorithm of the medical devices fails to correct an anomaly detected during the period of time.

Clause 19. The medical device resource management system of any one of Clauses 8 through 18, wherein the medical device resource management system is configured to update, delete, or download emergency protocol information via the communication interface.

Clause 20. A machine-implemented method, comprising: monitoring a plurality of medical devices for adjustments to the medical devices during a period of time; identifying, based on the monitoring, a plurality of the adjustments to the plurality of medical devices during the period of time; building a therapy profile associated with a patient and the medical devices based on the identified adjustments; identifying an adverse event pertaining the patient or a respective medical device of the plurality of medical devices; and after identifying the adverse event: generating, based on the therapy profile, a plurality of consecutive control signals to correct the adverse event, each being associated with a different one of the medical devices or a different user.

Clause 21. The machine-implemented method of Clause 20, wherein the method further includes receiving, from a plurality of user devices, information about roles of respective users associated with corresponding user devices; and displaying, based on the plurality of consecutive control signals, different protocol information at the corresponding user devices based on the roles of respective users.

Clause 22. A non-transitory machine-readable medium storing instructions thereon that, when executed by a device, cause the device to perform a machine-implemented method according to any one of Clauses 20 through 21.

Clause 23. The non-transitory machine-readable medium of Clause 22, wherein the device is an infusion control device.

Clause 24. An infusion control device configured to perform a method according to any one of Clauses 1 through 7.

### Further Consideration

It is understood that the specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, an ML assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A method for medical device resource management, the method comprising, under control of one or more processing devices:
monitoring a plurality of medical devices for adjustments to the medical devices during a period of time;
identifying, based on the monitoring, the adjustments to the plurality of medical devices during the period of time;
building a therapy profile associated with a patient and the medical devices based on the identified adjustments;
identifying an adverse event pertaining the patient or a respective medical device of the plurality of medical devices; and
after identifying the adverse event:
generating, based on the therapy profile, a plurality of consecutive control signals to correct the adverse event, each being associated with a different one of the medical devices or a different user.

2. The method of claim 1, further comprising:
sending each of the plurality of consecutive control signals to a different device associated with a different user.

3. The method of claim 1, wherein the monitoring the plurality of medical devices comprises monitoring medical devices that are associated with a procedure being performed during the period of time.

4. The method of claim 1, wherein a first control signal of the plurality of consecutive control signals causes a first message to be displayed on a first device, and a second control signal of the plurality of consecutive control signals causes a second message to be displayed on a second device at a different time than when the first message is displayed on the first device.

5. The method of claim 4, wherein the second control signal is sent to a device outside of an area associated with a procedure being performed during the period of time.

6. The method of claim 1, wherein the adverse event comprises a sensor failure,
wherein one of the consecutive control signals comprises a message for adjusting one of the medical devices.

7. The method of claim 1, wherein a first control signal of the plurality of consecutive control signals comprises a reset command configured to cause a restart an operation of a medical device.

8. A medical device resource management system, comprising:
a communication interface configured to exchange information with a plurality of medical devices, and to exchange information with at least one of a plurality of user devices;
a processor; and
a non-transitory computer readable medium including instructions that, when executed by the processor, cause the medical device resource management system to:
monitor the plurality of medical devices for adjustments to the plurality of medical devices during a period of time;
identify, based on the monitoring, a plurality of the adjustments to the plurality of medical devices during the period of time;
build a therapy profile associated with a patient and the plurality of medical devices based on the identified plurality of adjustments;
identify an adverse event pertaining the patient or a respective medical device of the plurality of medical devices; and
after identifying the adverse event:
generate, based on the therapy profile, a plurality of consecutive control signals to correct the adverse event, each being associated with a different one of the medical devices or a different user.

9. The medical device resource management system of claim 8, wherein the medical device resource management system is configured to send each of the plurality of consecutive control signals to a different device associated with a different user.

10. The medical device resource management system of claim 8, wherein monitoring the plurality of medical devices comprises monitoring medical devices that are associated with a procedure being performed during the period of time.

11. The medical device resource management system of claim 8, wherein a first control signal of the plurality of consecutive control signals causes a first message to be displayed on a first device, and a second control signal of the plurality of consecutive control signals causes a second message to be displayed on a second device at a different time than when the first message is displayed on the first device.

12. The medical device resource management system of claim 11, wherein the second control signal is sent to a device outside of an area associated with the procedure.

13. The medical device resource management system of claim 8, wherein the adverse event comprises a sensor failure,
wherein one of the plurality of consecutive control signals comprises a message for adjusting one of the medical devices.

14. The medical device resource management system of claim 8, wherein the medical device resource management system is configured to:
receive, from a plurality of user devices, information about roles of respective users associated with corresponding user devices; and
display, based on the plurality of consecutive control signals, different protocol information at the corresponding user devices based on the roles of respective users.

15. The medical device resource management system of claim 8, wherein the plurality of consecutive control signals causes display of protocol information after receiving a user selection of a therapy.

16. The medical device resource management system of claims 15, wherein the user selection of the therapy comprises a therapy for one of asystole, bradycardia, ventricular tachycardia, or ventricular fibrillation.

17. The medical device resource management system of claim 15 wherein the medical device resource management system is further configured to:
receive verifications of one or more actions performed in response to the user selection of the therapy; and
cause dynamic protocol information to be displayed based on the verifications of the one or more actions.

18. The medical device resource management system of claims 15, wherein the protocol information comprises emergency protocol information, and wherein the emergency protocol information is provided in accordance with a determination by the medical device resource management system that a close-loop control algorithm of the medical devices fails to correct an anomaly detected during the period of time.

19. The medical device resource management system of claim 8, wherein the medical device resource management system is configured to update, delete, or download emergency protocol information via the communication interface.

20. A machine-implemented method, comprising:
monitoring a plurality of medical devices for adjustments to the medical devices during a period of time;
identifying, based on the monitoring, a plurality of the adjustments to the plurality of medical devices during the period of time;
building a therapy profile associated with a patient and the medical devices based on the identified adjustments;
identifying an adverse event pertaining the patient or a respective medical device of the plurality of medical devices; and
after identifying the adverse event:
generating, based on the therapy profile, a plurality of consecutive control signals to correct the adverse event, each being associated with a different one of the medical devices or a different user.

21-24. (canceled)
